(19) United States
(12) Patent Application Publication
(10) Pub. No.: US 2017/0262949 A1
(43) Pub. Date: Sep. 14, 2017

(54) INVESTIGATIVE INTERVIEW MANAGEMENT SYSTEM

(71) Applicant: Daren JAY, Sydney, New South Wales (AU)

(72) Inventor: Daren JAY, Sydney, New South Wales (AU)

(21) Appl. No.: 15/329,923

(22) PCT Filed: Aug. 4, 2015
(86) PCT No.: PCT/AU2015/050437

§ 371 Date: Jan. 27, 2017

(30) Foreign Application Priority Data
Aug. 4, 2014 (AU) 2014902995

(51) Int. Cl. G06Q 50/18; G06Q 10/10; A61B 5/024; A61B 3/113; A61B 3/11; A61B 5/00; G09B 7/02; A61B 5/01

(52) U.S. Cl. CPC G06Q 50/18; G09B 7/02; G06Q 10/10; A61B 5/01; A61B 3/113; A61B 3/112; A61B 5/4803; A61B 5/024

(57) ABSTRACT

An investigative interview management system and in particular a processing system, computerised system, method and computer readable medium for implementing the same are disclosed. In one aspect, the processing system is configured to receive, from an interviewer, a request to generate an interview plan; generate, based on the request, an interview plan including a plurality of topics; receive, from one or more input devices during an interview between the interviewer and interviewee, input data; associate at least some of the input data with one or more of the topics of the interview plan; analyse at least some of the input data; and output, during the interview via one or more output devices, one or more notifications based on results of the analysis.

### CROSS-REFERENCE TO RELATED APPLICATIONS

The present application claims priority from Australian Provisional Patent Application No 2014902995 filed on 4 Aug. 2014, the content of which is incorporated herein by reference.

### FIELD OF INVENTION

The present invention relates to an investigative interview management system and in particular in relation to a computerized system, method and computer programs for implementing the same.

### BACKGROUND

Information carries more value when it is organized and managed in an efficient manner Information that is not organized and managed properly within an interview loses its evidential value. This necessitates an efficient information management system. The necessity of an efficient information management system is even more necessary in the field of forensics as information collected in investigative interviews (sometimes also called as forensic interviews) plays a crucial role in deciding the outcome of a case.

Investigative interviews involve collection of a lot of information. Due to the fluid nature of conversation within investigative interviews, there is a danger that information can be overlooked. Because of the fluid nature of the conversation, it can also be difficult to manage evidence and information strategically. This may result in loss of evidential value of the information collected.

Currently, there is no known technological solution that helps interviewers in strategically managing information and evidence prior to and during an investigative interview. At present, the management of forensic material at interview is typically done physically by interviewers who draft a typed written interview plan and then rely on hand or typed written notes within the interview to manage forensic material before, during and post-interview. However, the drawback with this approach is that a pre-prepared interview plan quickly loses relevance and strategic value once an interview commences because of the fluid nature of the interview. Written notes recorded during an interview can only be properly analyzed post-interview and analysis is only achieved within the interview to the cognitive capacity of the interviewer. Current processes have no forensic way of recording what notes are recorded and when and by whom.

It can be difficult for an interviewer to assess their progress in the interview. Generally, it an interview with a criminal suspect, a goal is to allow the criminal suspect to speak for a majority of the interview. However, inexperienced interviewers may not be aware that they are speaking too much in the interview.

Therefore, there is a need to overcome or alleviate one or more of the above-mentioned problems.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### SUMMARY

This summary is provided to introduce a selection of concepts in a simplified form that are further described below in the Preferred Embodiments. This summary is not intended to identify key features or essential features of the claimed subject matter, nor is it intended to be used to limit the scope of the claimed subject matter.

In a first aspect there is provided a processing system for investigative interview management, wherein the processing system is configured to:

receive, from an interviewer, a request to generate an interview plan;

generate, based on the request, an interview plan including a plurality of topics;

receive, from one or more input devices during an interview between the interviewer and interviewee, input data;

associate at least some of the input data with one or more of the topics of the interview plan;

analyse at least some of the input data;

output, during the interview via one or more output devices, one or more notifications based on results of the analysis.

In certain embodiments, the one or more notifications are indicative of one or more of the topics from the interview plan requiring investigation during the interview.

In certain embodiments, the input data is indicative of alleged facts provided by the interviewee which are input by the interviewer via the one or more input devices, wherein the processing system is configured to:

analyse at least some of the input data to determine one or more inconsistencies in relation to the alleged facts; and

output, via the one or more output devices, the notification indicative of the one or more inconsistencies.

In certain embodiments, the processing system is configured to analyse at least some of the input data using a record repository including a plurality of records to identify the one or more inconsistencies.

In certain embodiments, at least some of the records relate to collected evidence.

In certain embodiments, at least some of the records relate to alleged facts recorded from other interviewees.

In certain embodiments, the processing system is configured to issue one or more queries to a mapping processing system to identify one or more inconsistencies in relation to the alleged facts.

In certain embodiments, the request includes a selection of one or more records from the record repository to associate with the one or more topics of the interview plan, wherein the processing system is configured to retrieve at least a portion of the one or more records for presentation via the output device during the interview in response to a selection by the interviewer via the input device.

In certain embodiments, the one or more input devices includes a wearable computing device wearable by the interviewee, wherein at least some of the input data is indicative of timestamped physical characteristics sensed by the wearable computing device, wherein the processing system is configured to analyse the physical characteristics to detect a change in the one or more physical characteristics of the interviewee which coincided with at least some of the topics discussed during the interview, wherein the one or more topics which coincided with the changed in the one or more physical characteristics is determined based on timestamped topic focus data indicative of the interviewer's selection at least some of the one or more topics which received focus via a user interface output by the one or more output devices.

In certain embodiments, the one or more physical characteristics include a heart rate of the interviewee, wherein the processing system is configured to analyse the heart rate of the interviewee and output the one or more notifications indicative of the one or more topics that coincided with detected changes in the heart rate.

In certain embodiments, the one or more physical characteristics include a body temperature of the interviewee, wherein the processing system is configured to analyse the body temperature of the interviewee and output the one or more notifications indicative of the one or more topics that coincided with detected changes in the body temperature.

In certain embodiments, the one or more physical characteristics include eye characteristics, wherein the processing system is configured to analyse the eye characteristics of the interviewee and output the one or more notifications indicative of the one or more topics that coincided with detected changes in the eye characteristics.

In certain embodiments, the eye characteristics include at least one of eye movement and pupil dilation.

In certain embodiments, the processing system is a server processing system and wherein at least one of the one or more input devices and at least one of the one or more output devices are part of or connected to a client processing system in communication with the server processing system.

In certain embodiments, the processing system is configured to:

receive, via the one or more input devices, at least some of the input data indicative of real time audio data from at least a portion of the interview;

analyse the real time audio data by using, during the interview, a speaker diarization computer program to determine an amount of the interview spoken by the interviewer and an amount of the interview spoken by the interviewee; and

output, via the one or more output devices and during the interview, the one or more notifications indicative of the amount of the interview spoken by the interviewer and the interviewee.

In certain embodiments, the one or more notifications indicative of the amount of the interview spoken by the interviewer and the interviewee are presented as graphical indicia.

In certain embodiments, the one or more input devices includes a video camera, wherein the real time audio data is an audio component of real time video data captured by the video camera.

In a second aspect there is provided a system for investigative interview management, wherein the system includes a server processing system and a client processing system, wherein:

the server processing system is configured to:
receive, from the client processing system, a request to generate an interview plan;
generate, based on the request, an interview plan including a plurality of topics;
receive, from the client processing system, input data during an interview between the interviewer and interviewee;
associate at least some of the input data with one or more of the topics of the interview plan;
analyse at least a portion of the input data;
transfer one or more notifications to the client processing system based on results of the analysis;

the client processing system is configured to:
transfer the request to the server processing system;
transfer the input data to the server processing system; and
output, during the interview, the one or more notifications based on the results of the analysis.

In certain embodiments, the one or more notifications are indicative of one or more of the topics from the interview plan requiring investigation during the interview.

In certain embodiments, at least some of the input data is indicative of alleged facts provided by the interviewee which are input by the interviewer via the one or more input devices, wherein the server processing system is configured to:

analyse at least some of the input data to determine one or more inconsistencies in relation to the alleged facts; and

transfer, to the client processing system, the one or more notifications indicative of the one or more inconsistencies.

In certain embodiments, the server processing system is configured to analyse at least some of the input data using a record repository including a plurality of records to identify the one or more inconsistencies.

In certain embodiments, at least some of the records relate to collected evidence.

In certain embodiments, at least some of the records relate to alleged facts recorded from other interviewees.

In certain embodiments, the server processing system is configured to issue one or more queries to a mapping processing system to identify one or more inconsistencies in relation to the alleged facts.

In certain embodiments, the request includes a selection of one or more records from the record repository to associate with the one or more topics of the interview plan, wherein the server processing system is configured to retrieve at least a portion of the one or more records for presentation via the client processing system device during the interview in response to a selection by the interviewer via the client processing system.

In certain embodiments, the system includes a wearable computing device wearable by the interviewee during the interview, wherein the wearable computing device is configured to:

capture timestamped physical characteristics of an interviewee; and

transfer, to the server processing system, at least some of the input data indicative of:
the physical characteristics of an interviewee to the server processing system; and
timestamped topic focus data indicative of the at least some of the one or more topics which received focus via a user interface presented via the client processing system;

wherein the server processing system is configured to:
analyse the physical characteristics to detect a change in the one or more physical characteristics of the interviewee;
determine, based on the timestamped topic focus data, the one or more topics which coincided with change of the one or more physical characteristics of the interviewee during the interview;
generating and transferring, to the client processing system, the one or more notifications indicative of the detected change in the one or more physical characteristics of the interviewee and the one or more topics which coincided with the detected change in the one or more physical characteristics of the interviewee.

In certain embodiments, the wearable computing device is configured to sense a heart rate of the interviewee, wherein the server processing system is configured to analyse the heart rate of the interviewee and transfer the one or more notifications indicative of the one or more topics that coincided with detected changes in the heart rate to the client processing system for presentation to the interviewer during the interview.

In certain embodiments, the wearable computing device senses a body temperature of the interviewee, wherein the server processing system is configured to analyse the body temperature of the interviewee and transfer the one or more notifications indicative of the one or more topics that coincided with detected changes in the body temperature to the client processing system for presentation to the interviewer during the interview.

In certain embodiments, the wearable computing device captures eye characteristics of the interviewee, wherein the processing system is configured to analyse the eye characteristics of the interviewee and transfer the one or more notifications indicative of the one or more topics that coincided with detected changes in the eye characteristics to the client processing system for presentation to the interviewer during the interview.

In certain embodiments, the eye characteristics include at least one of eye movement and pupil dilation.

In certain embodiments, the system includes a microphone, wherein the server processing system is configured to:

receive, from the microphone, at least some of the input data indicative of real time audio data from at least a portion of the interview;

analyse the real time audio data by using, during the interview, a speaker diarization computer program to determine an amount of the interview spoken by the interviewer and an amount of the interview spoken by the interviewee; and

transfer, to the client processing system during progress of the interview, the one or more notifications indicative of the amounts of the interview spoken by the interviewer and the interviewee.

In certain embodiments, the one or more notifications indicative of the amounts of the interview spoken by the interviewer and the interviewee are presented by the client processing system as graphical indicia.

In certain embodiments, the system includes a video camera, wherein the server processing system is configured to receive the video data from the video camera and store the video data in a record repository.

In certain embodiments, the system includes a video camera including the microphone, wherein the server processing system is configured to receive at least some of the input data as video data from the video camera and use an audio component of the video data captured by the microphone as the real time audio data for analysis by the speaker diarization computer program.

In a third aspect there is provided a method for investigative interview management, wherein the method includes:

a processing system receiving, from an interviewer, a request to generate an interview plan;

the processing system generating, based on the request, an interview plan including a plurality of topics;

the processing system receiving, from one or more input devices during an interview, input data;

the processing system associating the input data with one or more of the topics of the interview plan;

the processing system analysing the input data;

the processing system output, during the interview via one or more output devices, one or more notifications based on results of the analysis.

In certain embodiments, the one or more notifications are indicative of one or more of the topics from the interview plan requiring investigation during the interview.

In certain embodiments, the input data is indicative of alleged facts provided by the interviewee which are input by the interviewer via the one or more input devices, wherein the method includes:

the processing system analysing at least some of the input data to determine one or more inconsistencies in relation to the alleged facts; and

the processing system outputting, via the one or more output devices, the notification indicative of the one or more inconsistencies.

In certain embodiments, the method includes the processing system analysing at least some of the input data using a record repository including a plurality of records to identify the one or more inconsistencies.

In certain embodiments, at least some of the records relate to collected evidence.

In certain embodiments, at least some of the records relate to alleged facts recorded from other interviewees.

In certain embodiments, the method includes the processing system issuing one or more queries to a mapping processing system to identify one or more inconsistencies in relation to the alleged facts.

In certain embodiments, the request includes a selection of one or more records from the record repository to associate with the one or more topics of the interview plan, wherein the method includes the processing system retrieving at least a portion of the one or more records for presentation via the output device during the interview in response to a selection by the interviewer via the input device.

In certain embodiments, the one or more input devices includes a wearable computing device wearable by the interviewee, wherein at least some of the input data is indicative of timestamped physical characteristics sensed by the wearable computing device, wherein the method includes the processing system analysing the physical characteristics to detect a change in the one or more physical characteristics of the interviewee which coincided with at least some of the topics discussed during the interview, wherein the one or more topics which coincided with the change in the one or more physical characteristics is determined based on timestamped topic focus data indicative of the interviewer's selection at least some of the one or more topics which received focus via a user interface output by the one or more output devices.

In certain embodiments, the one or more physical characteristics include a heart rate of the interviewee, wherein the method includes the processing system analysing the heart rate of the interviewee and outputting the one or more notifications indicative of the one or more topics that coincided with detected changes in the heart rate.

In certain embodiments, the one or more physical characteristics include a body temperature of the interviewee, wherein the method includes analysing the body temperature of the interviewee and outputting the one or more notifications indicative of the one or more topics that coincided with detected changes in the body temperature.

In certain embodiments, the one or more physical characteristics include eye characteristics, wherein the method includes the processing system analysing the eye characteristics of the interviewee and outputting the one or more notifications indicative of the one or more topics that coincided with detected changes in the eye characteristics.

In certain embodiments, the eye characteristics include at least one of eye movement and pupil dilation.

In certain embodiments, the processing system is a server processing system and wherein at least one of the one or more input devices and at least one of the one or more output devices are part of or connected to a client processing system in communication with the server processing system.

In certain embodiments, the method includes:

the processing system receiving, via the one or more input devices, at least some of the input data indicative of real time audio data from at least a portion of the interview;

the processing system analysing the real time audio data by using, during the interview, a speaker diarization computer program to determine an amount of the interview spoken by the interviewer and an amount of the interview spoken by the interviewee; and

the processing system outputting, via the one or more output devices and during the interview, the one or more notifications indicative of the amount of the interview spoken by the interviewer and the interviewee.

In certain embodiments, the method includes presenting the one or more notifications indicative of the amount of the interview spoken by the interviewer and the interviewee as graphical indicia.

In certain embodiments, the one or more input devices includes a video camera, wherein the real time audio data is an audio component of real time video data captured by the video camera.

In a fourth aspect there is provided a computer readable medium for configuring a processing system for investigative interview management, wherein the computer readable medium includes executable instructions which, when executed by a processing system, configure the processing system to perform the method of the third aspect and the related embodiments.

Other aspects and embodiments will be realised throughout the detailed description.

### BRIEF DESCRIPTION OF THE FIGURES

Example embodiments should become apparent from the following description, which is given by way of example only, of at least one preferred but non-limiting embodiment, described in connection with the accompanying figures.

FIG. 1 illustrates a functional block diagram of an example processing system that can be utilised to embody or give effect to embodiments;

FIG. 2 illustrates an example network infrastructure that can be utilised to embody or give effect to embodiments;

FIG. 3A illustrates a system diagram representing an example interview management system;

FIG. 3B illustrates a system diagram representing a further example of a interview management system including a server processing system and multiple client processing systems;

FIG. 4 illustrates an example screenshot of an interface of the client computer program where the interviewer inputs details for requesting generation of an interview plan;

FIG. 5 illustrates an example screenshot of an interface of the client computer program where the interviewer inputs details regarding the topics for the generation of an interview plan;

FIG. 6 illustrates an example screenshot of an interface of the client computer program depicting tiles representing topics of the generated interview plan;

FIG. 7 illustrates an example screenshot of an interface of the client computer program which enables the interviewer to input notes regarding a particular topic during the interview;

FIG. 8 illustrates an example screenshot of an interface of the client computer program which enables the interviewer to input an alleged temporal fact regarding a particular topic during the interview;

FIG. 9 illustrates an example screenshot of an interface of the client computer program which depicts a timeline of events for a matter;

FIG. 10 illustrates an example screenshot of an interface of the client computer program which depicts the data associated with one of the topics of the interview plan including a plurality of links to records of the record repository; and

FIG. 11 illustrates an example screenshot of an interface of the client computer program which depicts output data transferred from the server processing system to the client processing system based on analysis.

### DESCRIPTION OF THE PREFERRED EMBODIMENTS

The following modes, given by way of example only, are described in order to provide a more precise understanding of the subject matter of a preferred embodiment or embodiments. In the figures, incorporated to illustrate features of an example embodiment, like reference numerals are used to identify like parts throughout the figures.

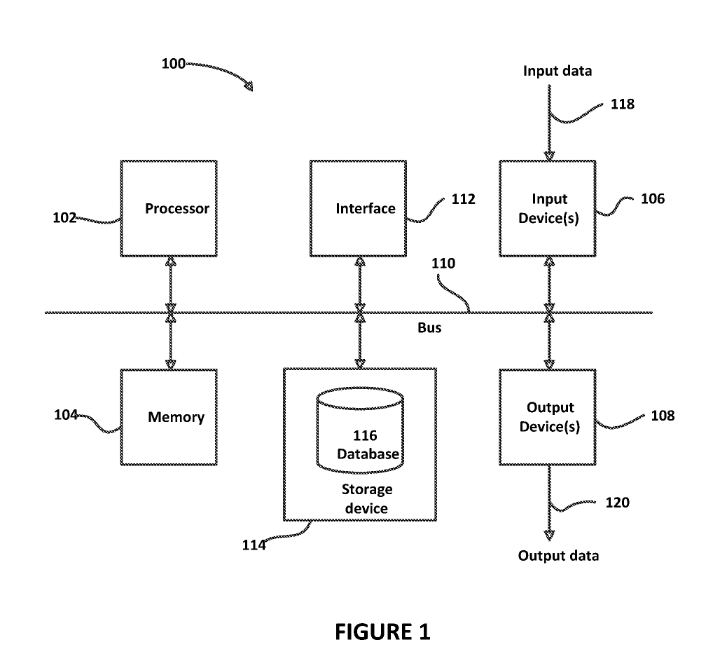

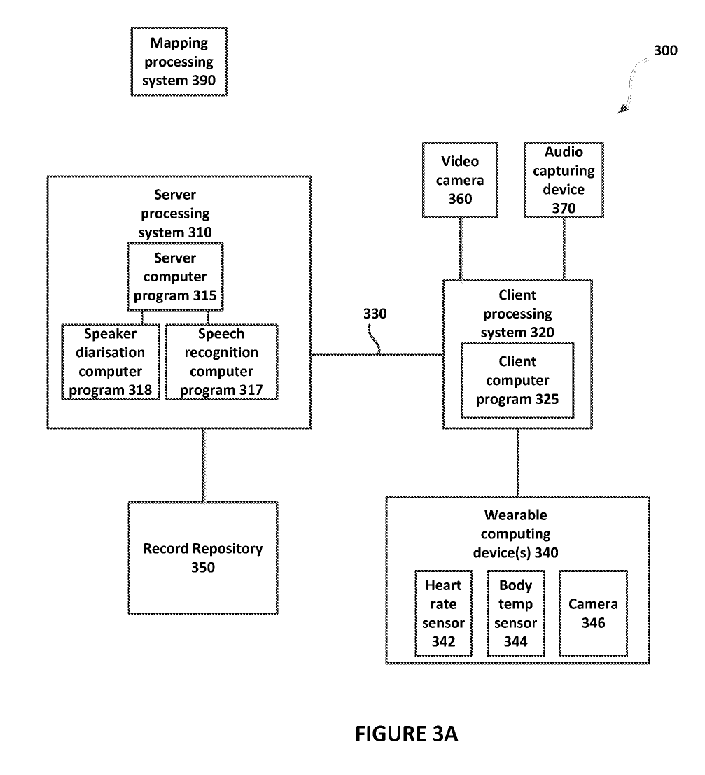

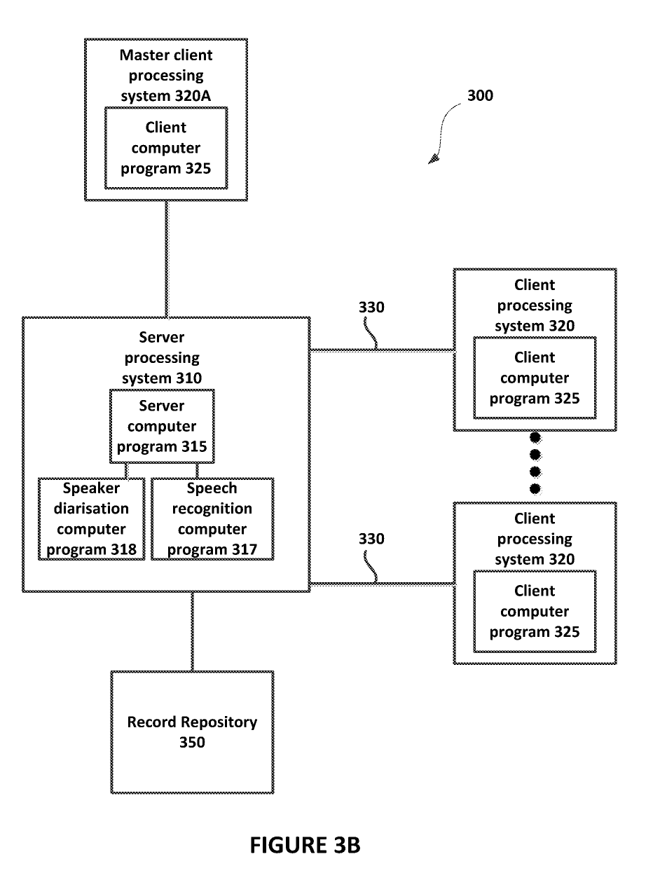

Referring to FIG. 3A there is shown a system diagram of system 300 for investigative interview management. The system 300 includes a server processing system 310 and a client processing system 320. The server processing system 310 together with the client processing system 320 form a distributed processing system. The client processing system 320 is preferably a mobile processing such as a tablet processing system which can be provided in the form of processing system 100 depicted in FIG. 1 and discussed herein, however it will be appreciated that other types of processing systems can also be utilised. The client processing system 320 is in data communication with the server processing system 310 via a computer network 330 such as a local private computer network and/or a public network such as the Internet.

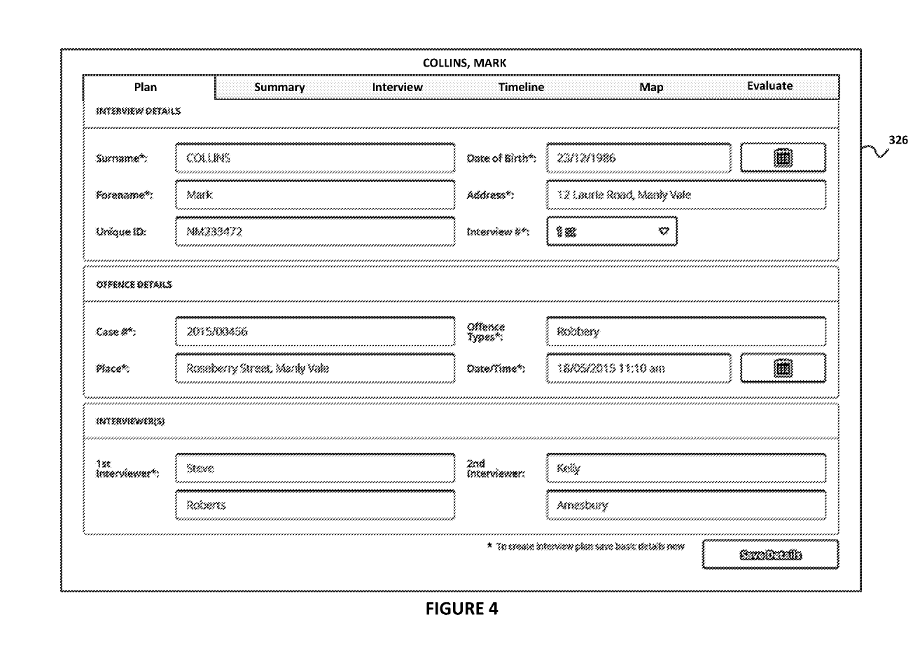

Generally, the server processing system 310 has a server computer program 315 installed in memory to configure the server processing system 310 to operate in the manner described herein. Additionally, the client processing system 320 can have a client computer program 325 (commonly known as an “app”) installed in memory to configure the client processing system 320 to operate in the manner described herein. The computer programs 315, 325 may be embodied in the form of a non-transitory computer readable medium having executable instructions stored thereon. Example screenshots of a user interface of the client computer program are shown in FIGS. 4 to 10.

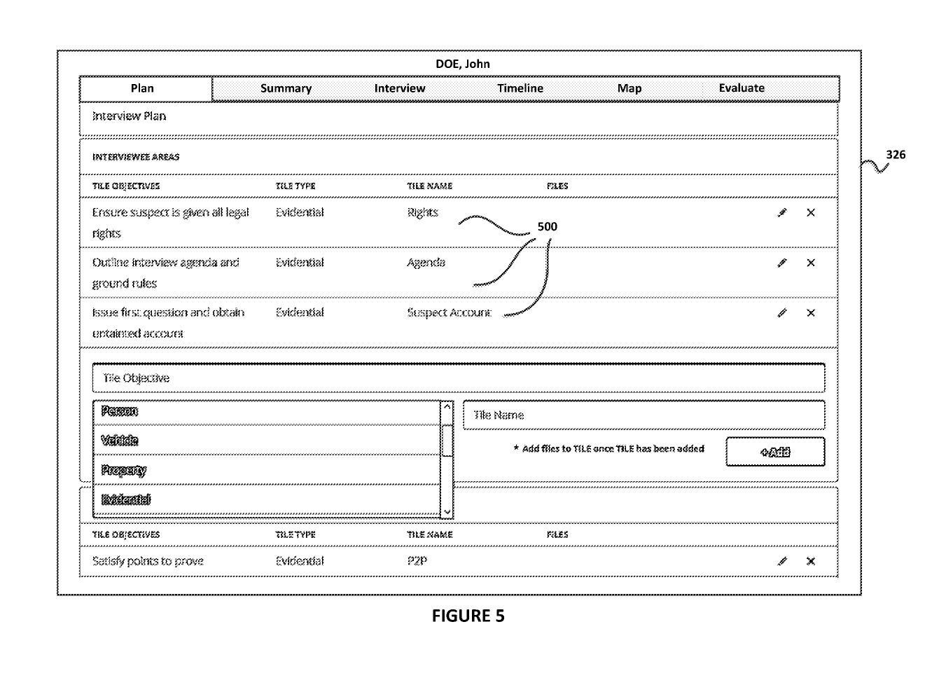

The server processing system 310 is configured by the server computer program 315 to receive, from an interviewer operating the client processing system 320, a request to generate an interview plan. An example of the information input by the interviewer via the interface 326 of the client computer program 325 for the request is shown in FIGS. 4 and 5, wherein FIG. 4 shows the interviewer able to enter details regarding the interview and FIG. 5 shows the interviewer able to define the one or more topics 500 of the interview. The server processing system 310 generates the interview plan including a plurality of topics 500 in response to the request. The server processing system 310 is additionally configured to receive, from one or more input devices of or associated with the client processing system 320, input data captured during the interview. The server processing system 310 associates at least some of the input data with one or more topics 500 of the interview plan. The server 310 then analyses at least some of the input data and outputs, to an output device of the client processing system 320, output data which can include one or more notifications based on results of the analysis. The one or more notifications can be indicative of one or more topics which require further investigation by the interviewer during the interview. However, as will be discussed herein, other output data can be presented via the interface 326.

It will be appreciated by those skilled in the art that the input and output device of the client processing system 320 for capturing and presenting the input data and the notification respectively may be the same component of the client processing system 320, namely a touch screen display or the like. It will also be appreciated that the system may include multiple input devices and multiple output device, as will be discussed.

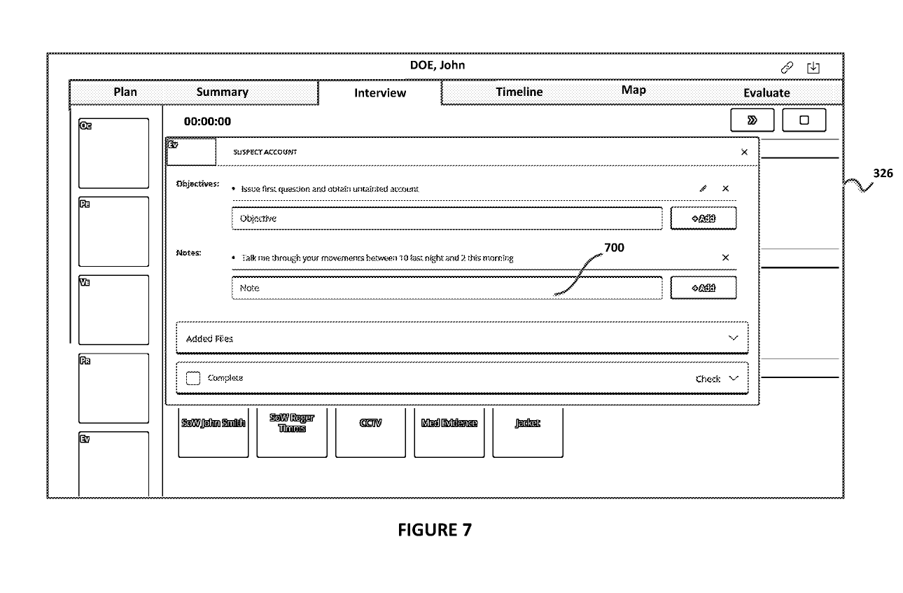

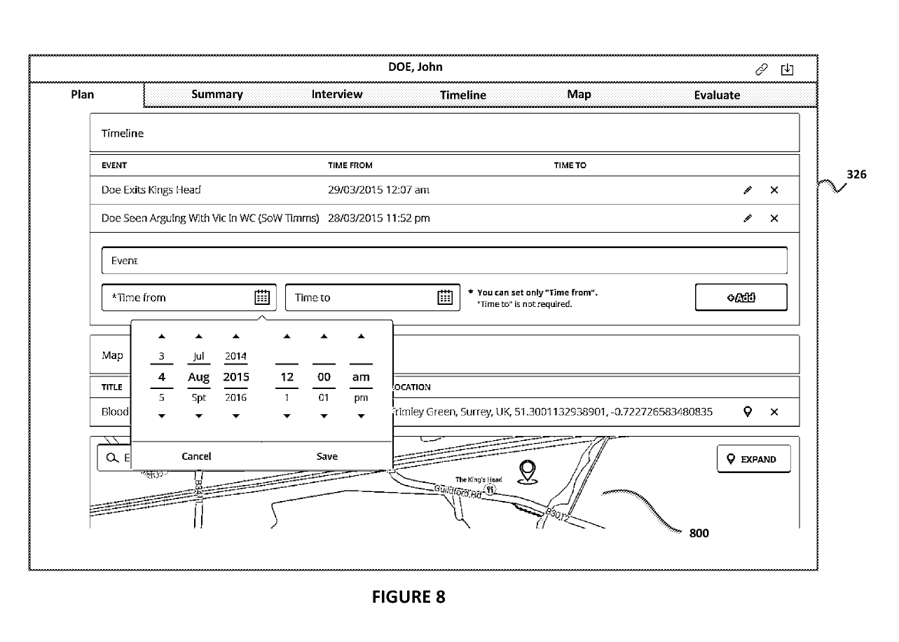

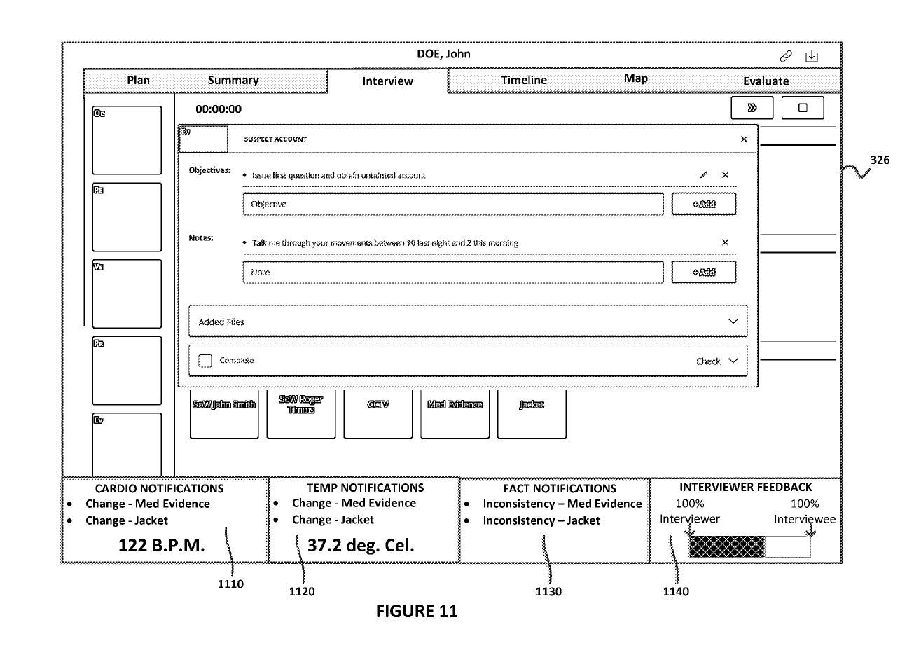

At least some of the input data can be indicative of alleged facts provided by the interviewee which are input by the interviewer via the input device of the client processing system 320. For example, as shown in FIGS. 7 and 8, the interviewer may enter notes 700 in relation to one of the topics 500. For example, the interviewee may indicate that they were located at a first location at 10 pm and a second location at 11 pm and that they travelled by foot between the first and second locations. The server processing system 310 can analyse this input data input by the interviewer to identify whether one or more inconsistencies exist in relation to the alleged facts. In this example, one or more queries can be generated by the server processing system and transferred to a location service provided by a mapping processing system 390, such as Google Maps or other similar services, via an application program interface (API) to determine whether it was possible for the interviewee to travel between the first and second location by foot. In the event the server processing system 310 determines that it was not possible based on the response data from the mapping processing system 390, the server processing system 310 may generate and transfer the one or more notifications 1130, as shown in FIG. 11, to the client processing system 320 indicating the identification of one or more inconsistencies which require further investigation during the interview. For example, the interviewer could confirm that dates and locations with the interviewee and then propose that the interviewees alleged facts are in fact false or incorrect. Data of which alleged facts could be compared against by the server processing system 310 could include mobile phone records, debit card transactions, GPS data, fuel cards, ROAM tags and any other data containing geo or time metadata.

The server processing system 310 can include or access a record repository 350 which stores therein a plurality of records. The record repository 350 can be provided in the form of a database. In one form, the record repository is provided in the form of cloud data storage. The input data received from the client processing system 320 can be stored by the server processing system 310 in the database. In addition, the server processing system 310 can analyse at least some of the input data using one or more of the records that are stored in the database. Continuing with the above example, at least some of the records may be indicative of evidence collected which place the interviewee at a third location at 10:30 pm. Therefore, the server processing system 310 may generate a query to the location service to determine whether it was possible for the interviewee to travel by foot from the first location at 10 pm to the third location by 10:30 pm, and then travel by foot from the third location at 10:30 pm to the second location at 11 pm. In the event that either portions of this query result in the identification of an inconsistency, the server processing system 310 generates the notification indicating that the interviewer should pursue further questioning in light of the identified inconsistency.

It will be appreciated that in the event multiple interviews are being conducted in relation to the same matter (e.g. multiple suspects in a crime), multiple client processing systems 320 can be in data communication with the server processing system 310, as shown in FIG. 3B, resulting in multiple streams of input data being stored to the database 350. In this arrangement, the inconsistencies between one interviewees alleged facts and another interviewees alleged facts can be identified by the server processing system 310 in a similar manner as described above resulting in one or more notifications being generated and presented to the relevant interviewers.

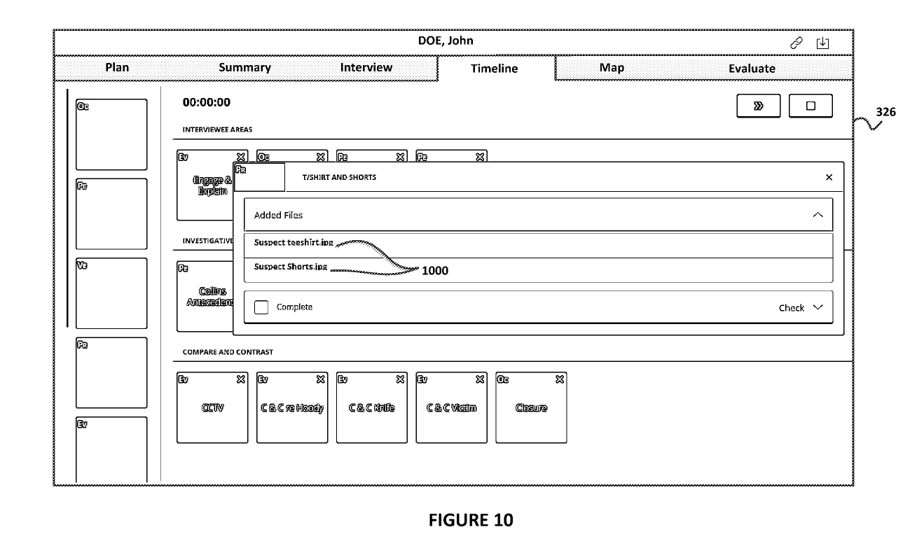

The interview plan is generated in the form of an interview data plan structure which stored in the database, wherein the interview data plan structure includes a plurality of topic data structures representing data associated with the respective topics. As shown in FIG. 10, each topic data structure can include or have associated therewith a plurality of links to one or more records from the record repository 350 as discussed below. The interview plan can be generated in response to the request based on a nominated selection of one or more records from the record repository 350. In particular, for a particular matter there may be a large number of records recorded in the database which may overwhelm the interviewer in the interview. Therefore, the interviewer can nominate a selection of the records from the record database for one or more of the topics to be discussed in the interview. Each topic 500 is effectively treated as a logical grouping of associated records for use by the interviewer to effectively plan and conduct the interview. The selected records are associated with the topic 500 via a link. Therefore, a topic may have a plurality of links 1000 to records in the record repository 350. In this manner, the client processing system 320 does not need to download all the selected records from the data repository 350 which can be problematic if insufficient memory is an issue. When the interviewer is conducting the interview and would like to access one of the records that have been nominated, the interviewer can select one of the topics from an interface presented via the client processing system 320 and then select one of the relevant links 1000. In response, the client processing system 320 sends a retrieval request to the record repository 350 via the server processing system 310, and a response is received by the client processing system 320 via the server processing system 310 indicative of the retrieved record. The retrieved record can then be presented via the interface of the client computer program running on the client processing system 320. It will be appreciated that particular records may be cached by the client processing system 320.

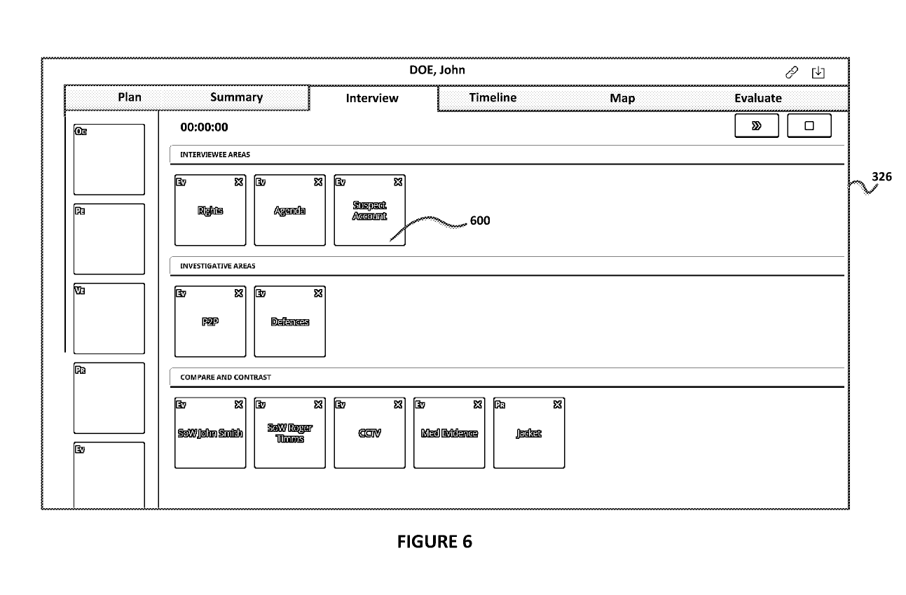

As shown in FIG. 6, the interface 326 of the client computer program 325 may present a number of tiles 600 arranged in a particular order defined in the generation of the interview plan. The interviewer can drag and rearrange the order of the tiles 600 on the interface 326 in order to better manage the information in response to the dynamic flow of the interview. In particular, the user can select one of the tiles 600 representing the current topic being discussed in the interview and drag the tile 600 to a new position in the interface to reflect the flow of the contents of the interview. The interviewer can additionally select the tile 600 to be presented with the links 1000 to the one or more records. Additionally, as shown in FIG. 7, input fields are presented in relation to one of the tiles 600 being selected which allows the interviewer to record the input data via the interface 326 of the client processing system 320. When a tile 600 is selected from the interface 326, the client processing system 320 generates timestamped topic focus data indicative of the interviewer's selection one of the tiles 600 representing one of the topics 500 discussed during the interview. The timestamped topic focus data can be transferred from the client processing system 320 to the server processing system 310 for use in further analysis as will be discussed below.

Referring to FIG. 3, the system can also include a number of additional or alternate input devices. In particular, the input device can include a wearable computing device 340 worn by the interviewee, wherein the input data provided by the input device is indicative of one or more physical characteristics of the interviewee. The input data can be transferred from the wearable computing device 340 to the client processing system 320 and then forwarded to the server processing system 310, or alternatively be forwarded to the server processing system 310 in another manner.

In one specific form, the wearable computing device 340 can be heart rate monitor 342, wherein the one or more physical characteristics indicated by at least some of the input data include a heart rate of the interviewee. The server processing system 310 can be configured to analyse the heart rate of the interviewee and output the one or more notifications 1120, as shown in FIG. 11, indicative of the one or more topics 500 that coincided with detected changes in the heart rate. The wearable computing device 340 in this form may be provided in the form of a wristband that includes a sensitive sensor to capture the user's heart rate however the wearable computing device 340 may be worn on other areas considered suitable. The one or more topics 500 that coincided with the detected changes in the heart rate can be determined by the server processing system 310 based on synchronising the timestamped topic focus data received from the client processing system with the timestamped sensed physical characteristics data.

In an additional or alternate form, the wearable computing device 340 can be provided in the form of a body temperature sensor 344 which captures input data indicative a body temperature of the interviewee. Similarly to the above arrangement with the heart rate, the server processing system 310 can be configured to analyse the body temperature of the interviewee and output the one or more notifications 1110, as shown in FIG. 11, indicative of the one or more topics 500 that coincided with detected changes in the body temperature using the timestamped topic focus data. Again, the wearable computing device 340 may be provided in the form of a wristband that includes a sensitive sensor to capture the user's body temperature however the wearable computing device 340 may be worn on other areas considered suitable. It should be understood that a single wristband to detect both the heart rate and body temperature can be utilised.

In another additional or alternate form, the input device can include a camera device 346 to capture the one or more physical characteristics including eye characteristics of the interviewee. In particular, the input device can be a head mounted camera device 346, such as Google Glass, wherein the server processing system 310 is configured to analyse the eye characteristics of the interviewee and output the notification indicative of the one or more topics that coincided with detected changes in the eye characteristics using the timestamped topic focus data. The eye characteristics can include at least one of eye movement and pupil dilation.

Metadata indicative of physiological data can be stored against interview audio/video data in order that instant analysis and interpretation of the physiological data based on the topics of conversation and/or audio of that conversation. The physiological data can be presented as a grade along a ‘heat meter’, as shown in FIG. 11, demonstrating visually how little or much physiological output was recorded whilst the specific topic tile/topic was open/received focus during the interview. The user can play back the audio/video recording of the audio/video data during post interview analysis wherein the metadata can be interpreted by the server processing system 320 to generate, during post interview review, synchronised visual feedback of the physiological data captured which is presented via the interface 326.

In an additional arrangement, the system 300 can include a video camera device 360 and/or an audio recording device 370 such as a microphone to capture video and/or audio of the interview which is transferred to the server processing system 310 for storage in the database. The video camera device 360 and/or the audio recording device 370 can be part of the client processing system 320 or alternatively be separate components of the system 300. The server processing system 310 can be configured to use speech recognition software to generate a textual representation of the interview based on this received data which is also stored in the database 350.

In one form, the server processing system can have stored in memory a speaker diarization computer program 318 which may be a separate computer program to the server computer program 315 or a part of the server computer program 315. In response to the server processing system 310 receiving audio data from the client processing system 320, the server processing system 310 analyses the real time audio data by using the speaker diarization computer program 318 to determine an amount of the interview spoken by the interviewer and an amount of the interview spoken by the interviewee. The server processing system then transfers, to the client processing system 320, the one or more notifications indicative of the amount of the interview spoken by the interviewer and the interviewee. In one form, as shown in FIG. 11, the amounts may be represented in the form of a graphic such as a marker 1140 on a bar. Depending upon the location of the marker on the bar, the interviewer can quickly discern feedback regarding their participation in the interview and potentially can take immediate action in response. Alternatively, textual information may be presented such as a percentage indicative of the proportion of the interview spoken by the interviewer or interviewee. Generally, the interviewer wishes to limit the amount of talking and participation in the interview, therefore the graphic/textual information presented via the client app can provide quick useful feedback to the interviewer when the interviewer may be concentrating on other investigative matters during the interview.

In one form, the speaker diarization computer program 318 uses a pre-trained speaker model for the interviewer which stored in memory, such as the database 350. The server computer program 315 can identify the pre-trained speaker model of the interviewer based on login details provided by the interviewer into the client app 325. As audio data of the interviewee may not have been previously analysed by the speaker diarization computer program 318, an untrained speaker model can initially be utilised by the speaker diarization computer program 318, wherein online/real time training of the interviewee speaker model occurs during the interview. During the interview, the interviewee speaker model is automatically adjusted to improve accuracy. In an optional form, the graphic/textual feedback is not presented via the client interface 326 until a threshold period of time (e.g. 5 or 10 minutes) has elapsed in order to only display reasonably accurate feedback to the interviewer. The interviewee speaker model can be stored in the database 350 such that in future interviews with the interviewee, the interviewee speaker model can be retrieved by the server processing system 310 based on input data indicative of the name of the interviewee being interviewed when generating the interview plan. It will be appreciated that the speaker diarization computer program 318 utilises a hybrid approach of using a combination of offline and online training for speaker diarisation. An example of a hybrid speaker diarization system which can be used for the current system is disclosed by Vaquero et al. in “A Hybrid Approach to Online Speaker Diarization”, the contents of which is herein incorporated by reference.

In one form, results of the speaker diarisation computer program 318 can be used in combination with a speech recognition computer program 317 enabling for the server computer program 315 to determine the verbal word totals for each of the interview participants. The graphic or textual indicia presented via interface 326 as shown in FIG. 11 may be indicative of a scale of average spoken word ratios between interviewer/interviewee based on the word count attributed toward the interviewer and interviewee. It will be appreciated that the feedback can be useful for post interview analysis for interviewers reviewing their techniques with mentors and the like.

It will be appreciated from above that multiple input devices can be utilised to provide various streams of input data to the server processing system 310 to identify topics of the interview that require further investigation. As the analysis occurs automatically at the server processing system 310 without requiring the interviewer to request the analysis, the interviewer can concentrate on conducting the interview and being prompted by the client processing system 320 if one or more topics of the interview require investigation.

In another arrangement as shown in FIG. 3B, the system 300 can include a master client processing system 320A in data communication with the server processing system 310. The master client processing system 320A may receive a stream of information collected by the server processing system 310 from a plurality of the client processing systems 320 used for managing multiple interviews substantially simultaneously. The master client processing system 320A may be operated by a person responsible for the operation of the interviews such as a head police investigator or the like. For example, in the event that the information presented via the master client processing system 320A indicates that an interviewee has indicated an alibi, the head police investigator can then arrange for this to be investigated whilst the interview is still proceeding. Information about the alibi could then be input by the master client processing system 320 or alternatively another client processing system 320 and stored in the record repository 350 such that it may be used for further questioning during the one or more interviews currently being conducted or to be conducted in the future. The master client processing system 320A can also be utilised by a head investigator to enable real-time decisions to be made regarding deployment of resources for situations such as search/recovery operations etc.

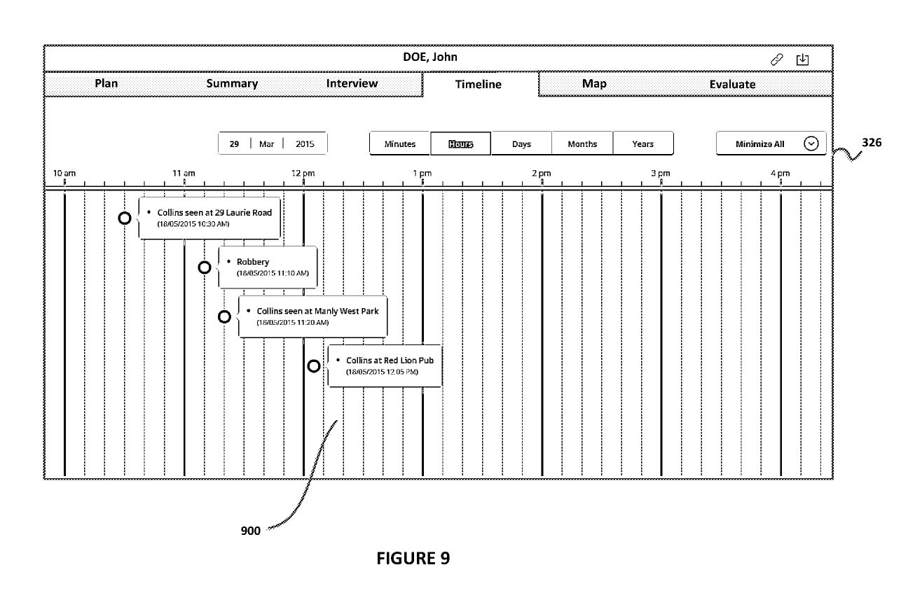

In certain embodiments, the interviewer may interact with the client processing system to request display of an timeline 900, as shown in FIG. 9, representing temporal events that have been input or stored in the database in relation to a particular investigation. The server processing system 320 may generate the timeline and transfer timeline data for presentation to the interviewer via the client processing system 320.

As shown in FIG. 8, events that occurred at various locations can be presented via a map interface 800 via the client interface 326, wherein the map interface can be generated by the mapping processing system 390 via one or more requests by the server processing system 310 via the respective API of the mapping processing system 390.

It will be appreciated from the above that a cloud hosted software application can be provided which utilises server technology and interfaces simultaneously with multiple client processing systems 320 and uses a novel software application to perform investigative interview management. The client processing systems 320 enables the user to manage information strategically in real-time. The system enables the user to plan, conduct and review the interviews. The system also enables the user to compile objectives of the interviews and capture time, geographic and documentary related information prior to the interview then utilise that information through the use of a tiling interface. The system can be used to digitally record audio and all software actions executed on client processing system 320 for the purposes of real-time and post-interview review and analysis via web based servers. Remote review also facilitates real-time and post-interview supervisory oversight and collaborative planning capabilities. The tiling interface is used as topic areas and can be pre-populated with generic data attributes which can be edited to further meet the specific user requirements. The system 300 enables the user to add related notes to the information provided by the interviewee via client processing system 320 during the interview. The system is designed to offer forensic value by permitting a first interviewer to input notes on a client processing system 320 which, through refresh functionality, will be updated via the server processing system 310 and immediately available to a second interviewer using a secondary client processing system 320. The system also enables the user to attribute material to mapping and timeline functions. Remote analysis of this information including timelines and/or topographical information is then able to be analyzed remotely during the interview and in real-time.

One advantage of the embodiment disclosed is it facilitates efficient and strategic management of information at all stages that is, prior to the interview, during the interview and post interview. This system 300 can be adapted for use in other fields too. For instance, embodiments can be adapted for using in medical field and also for the purposes of cross-examination by legal practitioners.

An example of a processing system suitable for use as a server processing system, client processing system, and/or wearable computing device is shown in FIG. 3. In particular, the processing system 100 generally includes at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 also can be provided for coupling the processing system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could include more than one distinct processing device, for example to handle different functions within the processing system 100.

Input device 106 receives input data 118 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can include, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, the at least one database 116 and/or the memory 104. The interface 112 may allow wired and/or wireless communication between the processing unit 102 and peripheral components that may serve a specialised purpose. The processor 102 receives instructions as input data 118 via input device 106 and can display processed results or other output to a user by utilising output device 108. More than one input device 106 and/or output device 108 can be provided. It should be appreciated that the processing system 100 may be any form of terminal, server, specialised hardware, or the like.

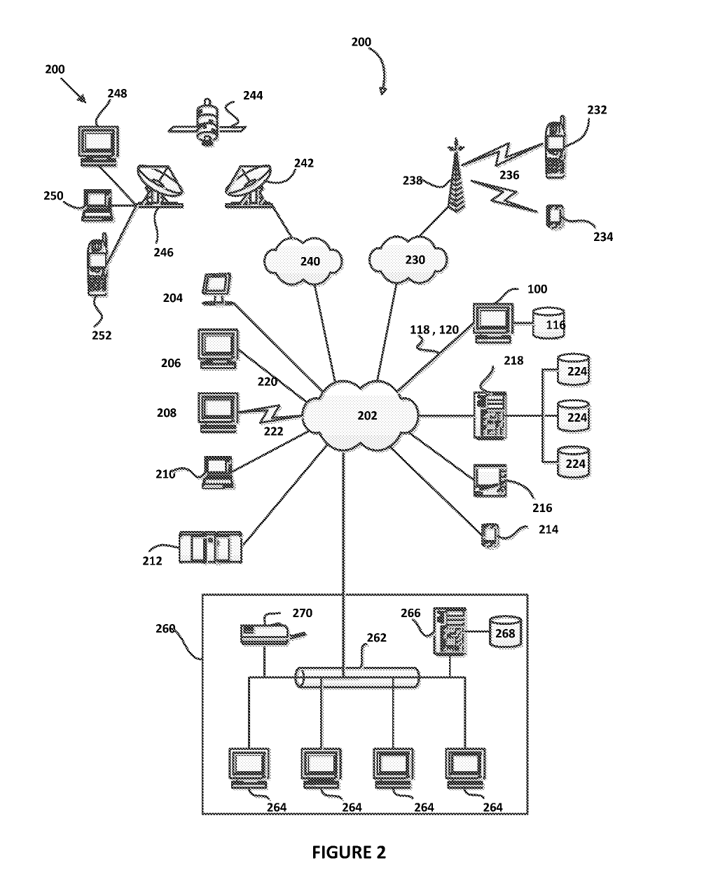

The processing device 100 may be a part of a networked communications system 200, as shown in FIG. 2. Processing device 100 could connect to network 202, for example the Internet or a WAN. Input data 118 and output data 120 could be communicated to other devices via network 202. Other terminals, for example, thin client 204, further processing systems 206 and 208, notebook computer 210, mainframe computer 212, PDA 214, pen-based computer 216, server 218, etc., can be connected to network 202. A large variety of other types of terminals or configurations could be utilised. The transfer of information and/or data over network 202 can be achieved using wired communications means 220 or wireless communications means 222. Server 218 can facilitate the transfer of data between network 202 and one or more databases 224. Server 218 and one or more databases 224 provide an example of an information source.

Other networks may communicate with network 202. For example, telecommunications network 230 could facilitate the transfer of data between network 202 and mobile or cellular telephone 232 or a PDA-type device 234, by utilising wireless communication means 236 and receiving/transmitting station 238. Satellite communications network 240 could communicate with satellite signal receiver 242 which receives data signals from satellite 244 which in turn is in remote communication with satellite signal transmitter 246. Terminals, for example further processing system 248, notebook computer 250 or satellite telephone 252, can thereby communicate with network 202. A local network 260, which for example may be a private network, LAN, etc., may also be connected to network 202. For example, network 202 could be connected with Ethernet 262 which connects terminals 264, server 266 which controls the transfer of data to and/or from database 268, and printer 270. Various other types of networks could be utilised.

The processing device 100 is adapted to communicate with other terminals, for example further processing systems 206, 208, by sending and receiving data, 118, 120, to and from the network 202, thereby facilitating possible communication with other components of the networked communications system 200.

Thus, for example, the networks 202, 230, 240 may form part of, or be connected to, the Internet, in which case, the terminals 206, 212, 218, for example, may be web servers, Internet terminals or the like. The networks 202, 230, 240, 260 may be or form part of other communication networks, such as LAN, WAN, Ethernet, token ring, FDDI ring, star, etc., networks, or mobile telephone networks, such as GSM, CDMA or 3G, etc., networks, and may be wholly or partially wired, including for example optical fibre, or wireless networks, depending on a particular implementation.

Many modifications will be apparent to those skilled in the art without departing from the scope of the present invention.

1. A processing system for investigative interview management, wherein the processing system includes one or more processors coupled to a memory, one or more input devices, and one or more output devices, wherein the one or more processors are configured to:
receive, from an interviewer, a request to generate an interview plan;
generate, based on the request, an interview plan including a plurality of topics;
receive, from one or more input devices during an interview between the interviewer and interviewee, input data;
associate at least some of the input data with one or more of the topics of the interview plan;
analyse at least some of the input data; and
output, during the interview via one or more output devices, one or more notifications based on results of the analysis.

2. (canceled)

3. The processing system according to claim 1, wherein the input data is indicative of alleged facts provided by the interviewee which are input by the interviewer via the one or more input devices, wherein the one or more processors are configured to:
analyse at least some of the input data to determine one or more inconsistencies in relation to the alleged facts; and
output, via the one or more output devices, the one or more notifications indicative of the one or more inconsistencies.

4. The processing system according to claim 3, wherein the one or more processors are configured to analyse at least some of the input data using a record repository including a plurality of records to identify the one or more inconsistencies.

5. The processing system according to claim 4, wherein at least some of the records relate to at least one of collected evidence and alleged facts recorded from other interviewees.

6. (canceled)

7. The processing system according to claim 3, wherein the one or more processors are configured to issue one or more queries to a mapping processing system to identify one or more inconsistencies in relation to the alleged facts.

8. (canceled)

9. The processing system according to claim 1, wherein the one or more input devices includes a wearable computing device wearable by the interviewee, wherein at least some of the input data is indicative of timestamped physical characteristics sensed by the wearable computing device, wherein the processing system is configured to analyse the physical characteristics to detect a change in the one or more physical characteristics of the interviewee which coincided with at least some of the topics discussed during the interview, wherein the one or more topics which coincided with the change in the one or more physical characteristics are determined based on timestamped topic focus data indicative of the interviewer's selection at least some of the one or more topics via a user interface of the processing system.

10. The processing system according to claim 9, wherein the one or more physical characteristics include at least one of:
a heart rate of the interviewee, wherein the one or more processors are configured to analyse the heart rate of the interviewee and output the one or more notifications indicative of the one or more topics that coincided with the detected change in the heart rate;
a body temperature of the interviewee, wherein the one or more processors are configured to analyse the body temperature of the interviewee and output the one or more notifications indicative of the one or more topics that coincided with detected change in the body temperature; and
an eye characteristic, wherein the one or more processors are configured to analyse the eye characteristic of the interviewee and output the one or more notifications indicative of the one or more topics that coincided with detected change in the eye characteristic.

11-12. (canceled)

13. The processing system according to claim 10, wherein the eye characteristic includes at least one of eye movement and pupil dilation.

14. The processing system according to claim 1, wherein the processing system is a server processing system and wherein at least one of the one or more input devices and at least one of the one or more output devices are part of or connected to a client processing system in communication with the server processing system.

15. The processing system according to claim 1, wherein the one or more processors are configured to:
receive, via the one or more input devices, at least some of the input data indicative of real time audio data from at least a portion of the interview;
analyse the real time audio data by using, during the interview, a speaker diarization computer program to determine at least one of an amount of the interview spoken by the interviewer and an amount of the interview spoken by the interviewee; and
output, via the one or more output devices and during the interview, the one or more notifications indicative of at least one of the amount of the interview spoken by the interviewer and the interviewee.

16. The processing system according to claim 15, wherein the one or more notifications indicative of at least one of the amount of the interview spoken by the interviewer and the interviewee is presented as graphical indicia.

17. (canceled)

18. A system for investigative interview management, wherein the system includes a server processing system and a client processing system, wherein:
the server processing system is configured to:
receive, from the client processing system, a request to generate an interview plan;
generate, based on the request, an interview plan including a plurality of topics;
receive, from the client processing system, input data during an interview between the interviewer and interviewee;
associate at least some of the input data with one or more of the topics of the interview plan;
analyse at least a portion of the input data; and
transfer one or more notifications to the client processing system based on results of the analysis; and
the client processing system is configured to:
transfer the request to the server processing system;
transfer the input data to the server processing system; and
output, during the interview, the one or more notifications based on the results of the analysis.

19-25. (canceled)

26. The system according to claim 18, wherein the system includes a wearable computing device wearable by the interviewee during the interview, wherein the wearable computing device is configured to:
capture timestamped physical characteristics of an interviewee; and
transfer, to the server processing system, at least some of the input data indicative of:
the physical characteristics of an interviewee to the server processing system; and
timestamped topic focus data indicative of the at least some of the one or more topics which received focus via a user interface presented via the client processing system;
wherein the server processing system is configured to:
analyse the physical characteristics to detect a change in the one or more physical characteristics of the interviewee;
determine, based on the timestamped topic focus data, the one or more topics which coincided with change of the one or more physical characteristics of the interviewee during the interview; and
generate and transfer, to the client processing system, the one or more notifications indicative of the detected change in the one or more physical characteristics of the interviewee and the one or more topics which coincided with the detected change in the one or more physical characteristics of the interviewee.

27. The system according to claim 26, wherein the wearable computing device is configured to sense or capture at least one of:
a heart rate of the interviewee, wherein the server processing system is configured to analyse the heart rate of the interviewee and transfer the one or more notifications indicative of the one or more topics that coincided with the detected change in the heart rate to the client processing system for presentation to the interviewer during the interview;
a body temperature of the interviewee, wherein the server processing system is configured to analyse the body temperature of the interviewee and transfer the one or more notifications indicative of the one or more topics that coincided with detected change in the body temperature to the client processing system for presentation to the interviewer during the interview; and
an eye characteristic of the interviewee, wherein the processing system is configured to analyse the eye characteristic of the interviewee and transfer the one or more notifications indicative of the one or more topics that coincided with detected change in the eye characteristic to the client processing system for presentation to the interviewer during the interview.

28-29. (canceled)

30. The system according to claim 27, wherein the eye characteristic includes at least one of eye movement and pupil dilation.

31. The system according to claim 18, wherein the system includes a microphone, wherein the server processing system is configured to:
receive, from the microphone, at least some of the input data indicative of real time audio data from at least a portion of the interview;
analyse the real time audio data by using, during the interview, a speaker diarization computer program to determine at least one of an amount of the interview spoken by the interviewer and an amount of the interview spoken by the interviewee; and
transfer, to the client processing system during progress of the interview, the one or more notifications indicative of at least one of the amount of the interview spoken by the interviewer and the interviewee.

32. The system according to claim 31, wherein the one or more notifications indicative of at least one of the amount of the interview spoken by the interviewer and the interviewee is presented by the client processing system as graphical indicia.

33-34. (canceled)

35. A method for investigative interview management, wherein the method includes:
a processing system receiving, from an interviewer, a request to generate an interview plan;
the processing system generating, based on the request, an interview plan including a plurality of topics;
the processing system receiving, from one or more input devices during an interview, input data;
the processing system associating the input data with one or more of the topics of the interview plan;
the processing system analysing the input data; and
the processing system output, during the interview via one or more output devices, one or more notifications based on results of the analysis.

36-51. (canceled)

52. Computer readable medium for configuring a processing system for investigative interview management, wherein the computer readable medium includes executable instructions which, when executed by a processing system, configure the processing system for investigative interview management, wherein the processing system includes a processor coupled to a memory, one or more input devices and one or more output devices, wherein the processor is configured to:
receive, from an interviewer, a request to generate an interview plan;
generate, based on the request, an interview plan including a plurality of topics;
receive, from one or more input devices during an interview between the interviewer and interviewee, input data;
associate at least some of the input data with one or more of the topics of the interview plan;
analyse at least some of the input data; and
output, during the interview via one or more output devices, one or more notifications based on results of the analysis.
